(19) United States
(12) Patent Application Publication
(10) Pub. No.: US 2012/0271096 A1
(43) Pub. Date: Oct. 25, 2012

(54) Disposable blood pump and catheter

(76) Inventors: Daniel Gelbart, Vancouver (CA); Samuel Victor Lichtenstein, Vancouver (CA); Kevin Mark Lichtenstein, Vancouver (CA)

(21) Appl. No.: 13/136,030

(22) Filed: Jul. 22, 2011

Related U.S. Application Data
(60) Provisional application No. 61/517,732, filed on Apr. 25, 2011.

(51) Int. Cl. A61M 1/10

(52) U.S. Cl. 600/16

(57) ABSTRACT

A blood pump for cardiac assist is made up from a catheter having a collapsible outer delivery tube and an inner suction tube that can be collapsible as well. The diameter of the tubing is reduced at the entry point to the body an a hole in the side of the delivery tube provides blood flow to the organs downstream from the catheter insertion point. The catheter is connected to an elastic tubing loop which forms a peristaltic pump when stretched over rotating arms equipped with rollers.

### FIELD OF THE INVENTION

The invention is in the medical field, and I particular in the field of blood pumps such as those used for heart assist or dialysis.

### BACKGROUND OF THE INVENTION

Many medical situations, such as heart failure, require assisting the heart or taking over completely the blood pumping function. Because of sterility concerns such devices tend to be single-use, or disposable. Current devices have two major shortcomings: high cost of the disposable part and a requirement for a large insertion hole in the artery or vein. Such pumps are typically inserted via the femoral artery for heart assist use, and via the arm or neck for dialysis use. Typical puncture size is 24 Fr (about 8 mm) for a 5 l/min pump, which is equivalent to the normal cardiac output. A 24 Fr puncture typically requires a cut-down and some surgical closure method, such as suturing, when the catheter is removed. Smaller punctures, such as 12 Fr or 14 Fr can be done without a cut-down or surgical closure. It is desired to minimize the closure size for a given pumping volume as well as have a low cost disposable part. It is also desired to have a system with a minimal blood volume in it, as the system is started by filling with a saline solution which dilutes the patient's blood. A smaller volume typically also needs a smaller surface area, this less chances of clotting. It is also desired to minimize blood damage, mainly hemolysis, be limiting the pressure, vacuum and shear rates. It is also desired to have a naturally fail-safe design, not relying on alarms, in order not to exceed the safe limits. The present invention meets all these requirements. Another requirement is not to cut off blood supply to the leg when femoral artery insertion is used. Traditionally the catheter used for blood delivery had to be hooked up to a blood pump. Since both the catheter and pump in the invention are made of flexible tubing, they can be combined into a single unit. All these requirements can be met by the disclosed blood pump and catheter.

### SUMMARY OF THE INVENTION

A blood pump for cardiac assist is made up from a catheter having a collapsible outer delivery tube and an inner suction tube that can be collapsible as well. The diameter of the tubing is reduced at the entry point to the body an a hole in the side of the delivery tube provides blood flow to the organs downstream from the catheter insertion point. The catheter is connected to an elastic tubing loop which forms a peristaltic pump when stretched over rotating arms equipped with rollers.

### BRIEF DESCRIPTION OF THE DRAWINGS

FIG. 1 is a general view of the system.

FIG. 2 is a cross section of the system.

FIG. 3 is a cross section of the system with provisions for femoral artery blood supply.

### DESCRIPTION OF THE PREFERRED EMBODIMENT

The preferred embodiment combines two previous devices, a blood pump and a delivery catheter, into a single unit. A suitable blood pump is disclosed by U.S. Pat. No. 3,784,323 and a collapsible catheter disclosed by US patent application 20090259089 having the same inventors as the current invention. Both documents are hereby incorporated by reference. The pump disclosed by U.S. Pat. No. 3,784,323 is different from conventional roller, or “peristaltic”, pumps by the fact that the flexible tubing is not sealed by pressing it with a roller onto a hard surface. Instead, it is sealed by stretching the tubing over a roller. This prevents local high pressure spots that can crush red blood cells. A regular roller pump can be used as well, by pressing the tubing against a soft surface, such as silicone rubber, by the rotating rollers. The catheter disclosed by patent application 20090259089 is different from current catheters as both the pressure side and the vacuum side are made from flexible thin walled tubing and therefore fully collapsible to a small diameter. Being collapsible allows inserting the catheter through a small puncture. In an alternate embodiment only the pressure side is made collapsible. Once inserted the catheter is expanded by being pressurized. The whole length of the catheter is expanded, to offer low resistance to blood flow, except for a very short section, about 1-5 cm long, at the point on entering the artery or vein. Since the unexpanded section is very short it does not significantly lower blood flow. A novel structure uses a set of pressurized outside tubes (the delivery side) to strengthen the inner suction tube (the suction side). The pump and catheter can be connected into a single disposable part, thus eliminating any connections between the pump and catheter. Any hook-up of blood lines has to be done with extreme care because of the fear of air embolism which can be fatal. A single unit also has a smaller volume, reducing blood dilution by the saline solution used to fill the device before use.

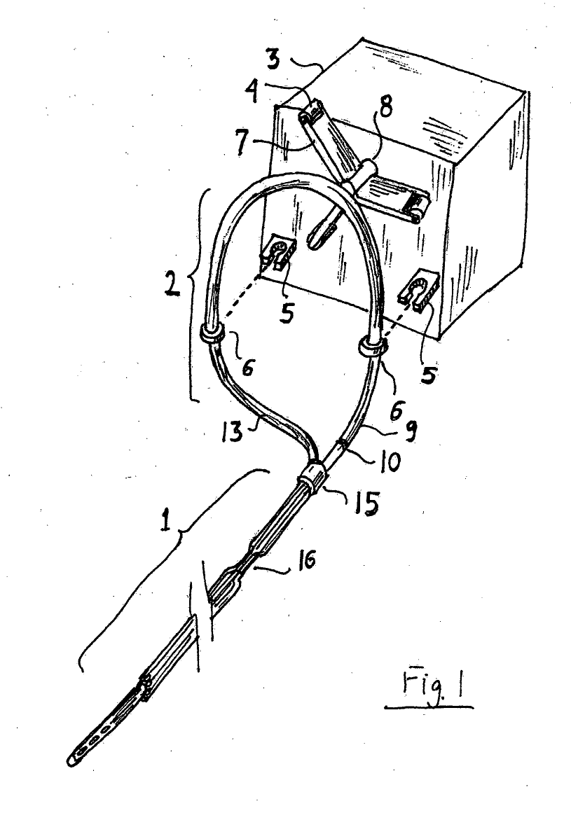

Referring now to FIG. 1, a single use flexible catheter 1 is attached to a flexible tubing loop 2 via connections 10 and 15. Suction side 9 is connected to the suction tube 22 of catheter 1 via connector 10. Pressure side 13 is connected to delivery tubes 14 via connector 15. Once pressurized by the pump, catheter 1 expands along its length except for entry point 16 which stays smaller. Typically catheter 1 will expand to an OD of 24 Fr-30 Fr while point 16 will expand only to 14 Fr-18 Fr to allow a smaller puncture in artery wall. Tubing loop 2 has two rings 6 for temporary attachment to holders 5 on pump 3. Clearly other means of attachment can be used, such as magnetic, temporary fasteners such as Velcro, clamping etc. Pump 3 has a rotating shaft 8 carrying multiple arms 7, each one with a free-spinning roller 4. The number of arms can be from 2 to over 10; however 3-6 are preferred.

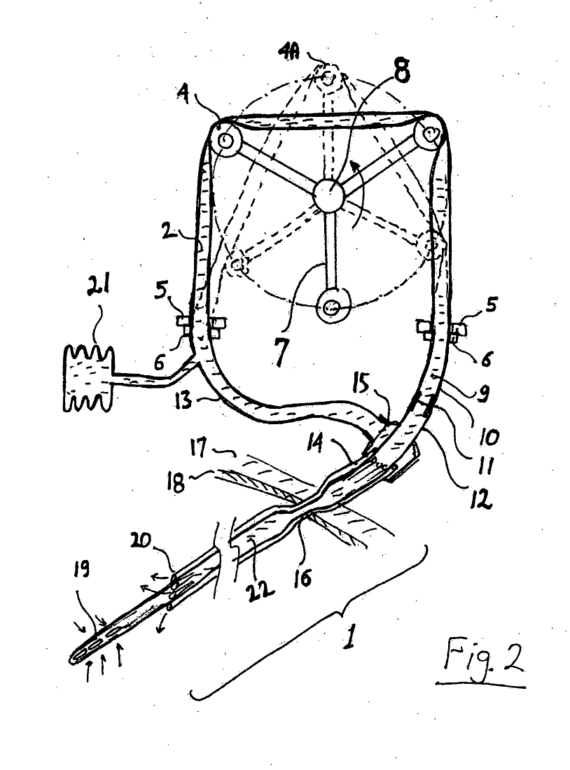

Referring now to FIG. 2, tubing loop 2 is sealed off, by stretching, at the points is touches rollers 4. Rings 6 are placed bellow holders 5 and allow tubing 2 to be stretched without coming off holders 5. The position of the roller 4 after arms 7 rotated is shown by dotted lines as roller 4A. The pump operation is similar to a standard roller pump. The soft “pinch-off” prevents blood damage. The pump provides semi-pulsatile flow. If more continues flow is desired the tubing itself can be used as an elastic reservoir or an additional elastic reservoir 21 can be added. The catheter is inserted in the collapsed state, filled with saline, through tissue 17 and vessel wall 18. Since all the tubing in catheter 1 are thin-walled the collapsed state can be quite small, typically below 18 Fr for a 5 liter/minute pump and below 9 Fr for a 1 liter/minute pump. As the pump inflates the delivery tubes 14 it supports the suction tube 22. For left ventricular assist the discharge point 20 is in the aorta and suction point 19 is in the left ventricle, i.e. the suction tube extends beyond the delivery tube. For left ventricular assist the last section of the suction tube 22, near discharge point 19, needs to be self-supporting against the outside pressure. The pressure differences in this section is low, typically below 10 mmHg, because it is at the end of the catheter. Tube loop 2 can be made of any blood compatible elastomer such as silicone rubber or polyurethane. Catheter 1 an be made from any blood compatible material that can be extruded into thin walled tubing, such as materials currently used to make stent balloons. Catheter 1 can be made from heat-shrinkable material. This allows the formation of neck 16 simply by heat shrinking the extruded tube. It is desired to add partitions or a divider inside the suction tube 22 in the area of neck 16 in order to reduce turbulent flow. The divider, typically a molded rigid insert, can be added to the extruded catheter before or after heat shrinking. This is detailed in patent application 20090259089. The complete design can be scaled to smaller sizes to be used on infants or as a dialysis catheter.

The inside of the tubing and catheter can be treated to minimize clotting by an anti-coagulation coating such as heparin or texturing the surface. A super-hydrophobic or super-hydrophilic coating may be used as well as an anti-clotting coating. When the suction tube 22 is made non-collapsible it should be reinforced by a metal wire embedded in a thin plastic wall. Good results were achieved with spring tempered stainless steel wires about 0.1 mm in diameter embedded in a 0.2 mm thick wall. The art of building catheters with wire reinforcement is well known and such tubes are commercially available.

Blood is more tolerant to pressure than to vacuum so the design optimizes the flow for a given size of entry point 16 by using a lower area for the pressure side than the suction side. Typical pressures used are 600-2000 mmHg with typical suction vacuum of 300-600 mmHg. In order to avoid high roller pressure in the roller pump, which could damage red blood cells, the high pressure can be built up by using multiple loops of tube 2 wound around the same set of rollers 4. By the way of example, if 1200 mmHg pressure and 300 mmHg suction are needed and tube 2 is looped around rollers 4 three times, the pressure differential per loop is only 400 mmHg and suction differential only 100 mmHg. Such low differentials allow to use the pump with tube not fully occluded at contact points with rollers, as leakage flow goes down with decreased pressure drop. This further reduces blood damage. In general the maximum pressure possible, per stage, is determined by the loop tension while the maximum suction possible is determined by the wall thickness of loop 2, since the elastic force of the tube wall allows it to stay round, resisting the suction force trying to collapse the tube. The pumping volume is determined by pump rotational speed, loop diameter and hose inside diameter.

Alternate ways of peristaltic action in loop 2 can be used as well, such as sequentially expanding air or gas bags. The advantage of using air bags instead of pump rollers is that the pump can be operated from a device similar to current balloon pumps.

The figures and descriptions show a pump inserted in the left ventricle or “left side assist”. The same design can also be used in the right ventricle as a “right side assist”. The required change is making delivery tube 14 extend beyond suction tube 22. The tip of tube 22 is connected to the outside of the longer tube 14 via an opening in the side of tube 14.

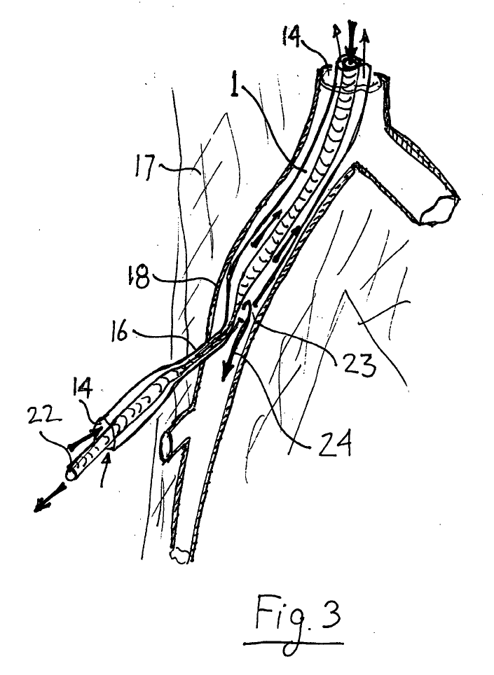

The section of the catheter inside the femoral artery (or other major arteries) has to be designed to maximize flow while minimizing occlusion of the artery, which could cause ischemia in the organs downstream from the catheter. In one embodiment the section of the catheter inside the artery after entry point 16 is tapered to match the natural tapering of the artery, typically expanding from 5 m-6 mm near entry point to 8 mm about 250 mm further along catheter. In the preferred embodiment the catheter is larger an is allowed to nearly or fully block the femoral artery, as seen in FIG. 3. A small opening 23 allows a blood stream 24 to profuse the organs, such as the leg, downstream from thee catheter. The size of opening 23 can be as small as 2-3 mm as the blood flow required is low, typically 50-100 cc/min.

The catheter as shown in FIG. 2 will discharge the blood into the aorta in a direction opposite to the normal flow. Normally this will not cause a problem, as once the aorta is pressurized the blood will flow towards lower pressure areas. If desired the discharge points 20 can be pointed sideways, by cutting slots into the pressure tube 14, or even pointed backwards by louvered slots.

By the way of example a catheter and pump combination according to the invention was built and tested. The inner tune was non-collapsible and reinforced by a spring made of 0.1 mm spring tempered stainless steel wire with both inside and outside walls being smooth. Total catheter length from entry point 16 to tip 19 was 70 cm. Delivery point 22 was 50 cm from entry point 16. The expandable sleeve delivery tube 14 was tapered from 5.6 mm at point 16 to 8 mm at 250 mm inside the body, after that expanded to 12 mm. All dimensions are when inflated. Delivery tube was made of 0.05 mm polyethylene. The hole 23 was about 1 mm. The suction tube had an ID of about 4.7 mm, with a narrowing to 4 mm for a length of 30 mm at the entry point 16. A cross shaped plastic divider, 30 mm long, was placed in the inner tube at the narrowed section to limit Reynold's number to below 3000. The delivery tube had an inside diameter of about 5.4 mm and an outside diameter of 5.6 mm at entry point 16. Tubing was shaped by stretching a heated tube. The catheter was tested with a 44% glycerin/56% water mixture, to simulate the viscosity of blood. A flow of 5 liter/minute was achieved at a pressure of 1000 mmHg and a suction vacuum of 400 mmHg. In the section outside the patient's body, between connector 15 and entry point 16, the tubing is not restricted in size. The pump loop 2 comprised of two turns of soft silicone rubber hose with an ID of 9.5 mm and an OD of 16 mm (McMaster-Carr part number 51135K37 purchased from www.mcmaster.com). While the examples in this disclosure are mainly for ventricular assist, the same device can be used as a high flow dialysis catheter or any other application requiring pumping fluids in and out of the body via a small puncture. The design is easily scalable over a wide range of sizes and flow rates.

1. A disposable blood pump comprising of a loop of flexible hose connected to a catheter having a suction tube inside a delivery tube, wherein at least said delivery tube is collapsible and is inflated after insertion into the body by the pressure of the blood pumped into the catheter.

2. A disposable blood pump comprising of a loop of flexible hose connected to a catheter having a suction tube inside a delivery tube, wherein both suction tube and delivery tube are collapsible, said delivery tube is inflated after insertion into the body by the pressure of the blood pumped into the catheter, and said suction tube is supported by the inflated delivery tube.

3. A disposable catheter for pumping blood into an artery having a smaller diameter section at the point of entry into the body, a suction tube surrounded by a collapsible outer sleeve inflated by blood flow, and an opening in said sleeve proximate to point of entry to maintain the blood supply to organs downstream from said catheter.

4. A blood pump as in claim 2 wherein the suction tube is formed by a plurality of delivery tubes bonded together.

5. A cardiac assist pump as in claim 1 wherein said flexible hose loop forms a part of a peristaltic pump.

6. A pump as in claim 2 wherein said flexible hose loop forms a part of a peristaltic pump.

7. A pump as in claim 1 wherein at least the suction tube includes a divider in order to reduce turbulent flow.

8. A pump as in claim 2 wherein at least the suction tube includes a divider in order to reduce turbulent flow.

9. A catheter for pumping blood as in claim 3 wherein at least the suction tube includes a divider in order to reduce turbulent flow.

10. A pump as in claim 1 wherein said loop of flexible hose is used in a multi-stage roller peristaltic pump by looping it several times around pump rollers.

11. A pump as in claim 2 wherein said loop of flexible hose is used in a multi-stage roller peristaltic pump by looping it several times around pump rollers.

12. A disposable cardiac assist pump as in claim 1 wherein the suction tube operates at a suction of 300-600 mmHg, the delivery tube operates at a range of 600-2000 mmHg and the flow rate is in the range of 1-5 liters per minutes.

13. A pump as in claim 1 wherein the hose loop is stretched over rotating rollers, forming a peristaltic pump.

14. A pump as in claim 2 wherein the hose loop is stretched over rotating rollers, forming a peristaltic pump.

15. A pump as in claim 1 wherein the suction tube extends beyond the delivery tube.

16. A pump as in claim 2 wherein the suction tube extends beyond the delivery tube.

17. A catheter as in claim 3 wherein the suction tube extends beyond the delivery tube.

18. A pump as in claim 1 wherein the delivery tube extends beyond the suction tube.

19. A pump as in claim 2 wherein the delivery tube extends beyond the suction tube.

20. A catheter as in claim 3 wherein the delivery tube extends beyond the suction tube.
